(19) United States
(12) Patent Application Publication
(10) Pub. No.: US 2009/0088403 A1
(43) Pub. Date: Apr. 2, 2009

(54) A3 ADENOSINE RECEPTORS AS TARGETS FOR THE MODULATION OF CENTRAL SEROTONERGIC SIGNALING

(76) Inventors: RANDY BLAKELY, Brentwood, TN (US); Chong-Bin Zhu, Nashville, TN (US); William Hewlett, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 12/116,629

(22) Filed: May 7, 2008

Related U.S. Application Data
(60) Provisional application No. 60/916,481, filed on May 7, 2007.

(51) Int. Cl. A61K 31/7076; A61K 31/4422

(52) U.S. Cl. 514/46; 514/356

(57) ABSTRACT

The present invention relates to the use of adenosine type 3 receptor (A3R) modulators to modulate serotonin transporter (SERT) function in vivo. In particular, antagonists of A3R can be used to inhibit A3R-dependent upregulation of SERT, for example, as antidepressants.

This application claims benefit of priority to U.S. Provisional Application Ser. No. 60/916,481, filed May 7, 2008, the entire contents of which are hereby incorporated by reference.

The government owns rights in the present invention pursuant to grant number DA07390 from the National Institutes of Health.

### BACKGROUND OF THE INVENTION

1. Field of the Invention

The present invention relates generally to the fields of neurobiology and genetics and pharmacology. More particularly, it concerns the use of A3R receptor-directed activators and antagonists as modifiers of neuronal serotonin transporters. These agents may be used in the treatment of disorders linked to altered brain serotonin availability including depression, anxiety disorders, autism and obsessive-compulsive disorder.

2. Description of Related Art

Central serotonin (5-hydroxytryptamine, 5-HT) signaling is critical to thermoregulation, appetite, sexual drive and mood. Multiple mechanisms contribute to the presynaptic control of serotonergic signaling including the synthesis, storage, release, and inactivation of 5-HT. In the CNS and periphery, 5-HT inactivation after release is accomplished via the antidepressant-sensitive 5-HT transporter (SERT), a presynaptic membrane protein. SERT is a member of the Na⁺/Cl⁻-dependent solute transporter family (SLC6A4) (Blakely et al., 1991; Hoffman et al., 1991; Ramamoorthy et al., 1993), and is related most closely to dopamine and norepinephrine transporters (DAT and NET, respectively). Targeted disruption of the murine SERT gene leads to a disruption of presynaptic 5-HT homeostasis (Bengel et al., 1998; Murphy et al., 2004) and is accompanied by anxiety-related behavioral changes (Holmes et al., 2003; Jennings et al., 2006). Human SERT gene variants have been linked to mental disorders including autism, depression, anxiety, and Obsessive-Compulsive Disorder (OCD) (Caspi et al., 2003; Murphy et al., 2003; Ozaki et al., 2003; Sutcliffe et al., 2005). Medications that treat facets of these disorders primarily target SERT (Barker and Blakely, 1995; Carrasco and Sandner, 2005; Serebruany, 2006) increasing extracellular 5-HT levels and augmenting postsynaptic 5-HT signaling, though their secondary targets may contribute to clinical side effects. The prevalence of 5-HT associated disorders underscores the importance of gaining a better understanding of the natural processes governing SERT regulation in vivo.

Previous studies demonstrate that SERTs are tightly controlled by multiple signaling pathways including G-protein coupled receptor (GPCR)-linked pathways (Blakely et al., 2005; Jayanthi and Ramamoorthy, 2005). Activation of protein kinase C (PKC) triggers a decrease in both SERT activity and surface expression in cultured cell lines (Qian et al., 1997; Ramamoorthy and Blakely, 1999), platelets (Jayanthi et al., 2005; Carneiro and Blakely, 2006), and nerve terminals (Samuvel et al., 2005), events linked to changes in membrane distribution (Jayanthi et al., 2004) and destabilization of SERT/associated protein complexes (Bauman et al., 2000; Quick, 2002; Carneiro and Blakely, 2006). In contrast, protein kinase G (PKG)- and p38 mitogen activated protein kinase (MAPK)-linked pathways support a rapid increase in SERT surface expression and function (Miller and Hoffman, 1994; Zhu et al., 2004a; Zhu et al., 2004b; Zhu et al., 2005). There is only limited information as to the specific receptors that utilize these pathways in vivo. Chronoamperometry studies suggest a role of presynaptic 5-HT, receptors in amplifying 5-HT clearance in rat hippocampus (Daws et al., 2000), through as yet unknown pathways. Ansah and coworkers demonstrated activity of presynaptic α2 adrenergic receptors in downregulation of SERT activity in mouse brain synaptosomes and slices (Ansah et al., 2003). Histamine receptor stimulation has been reported to induce an increase of 5-HT uptake linked to PKG activation in platelets (Launay et al., 1994); currently evidence that this pathway regulates SERT in the CNS is lacking. Miller and Hoffman (1994) first noted activity of adenosine receptors (ARs) in stimulating SERT activity via a cGMP linked pathway in rat basophilic leukemia (RBL-2H3) cells in vitro. Interestingly, an in vivo microdialysis study has noted the ability of AR-targeted drugs to alter extracellular 5-HT levels in rat hippocampus (Okada et al., 1999), suggesting that CNS parallels may exist for AR modulation of SERT activity. Recently, the inventors used RBL-2H3 cells and AR/SERT co-transfected Chinese Hamster Ovary (CHO) cells to further define an A₃AR-linked pathway that supports upregulation of SERT activity, establishing involvement of two distinct pathways: one, a PKG-dependent pathway linked to enhanced SERT surface trafficking and a second, supported by p38 MAPK, associated with a trafficking-independent process that enhances SERT catalytic activity by reducing the 5-HT Km (Zhu et al., 2004a; Blakely et al., 2005; Zhu et al., 2005). Whereas the latter pathway has been established to be targeted by inflammatory cytokines in CNS preparations (Zhu et al., 2006), receptors that initiate PKG-dependent SERT regulation in the brain are unknown. Thus, additional studies are required to exploit this target in the context brain-related disorders.

### SUMMARY OF THE INVENTION

Thus, in accordance with the present invention, there is provided a method of modulating serotonin transporter (SERT) function in a native brain preparation or tissue comprising reducing SERT activity with an antagonist of adenosine type 3 receptor (A3R) function. The antagonist may be MRS-1191, MRS-1523, or VUF5574. The method may further comprise measuring 5-HT uptake in the native brain preparation or tissue prior to modulation by SERT by the A3R agonist or antagonist, or measuring 5-HT uptake in the native brain preparation or tissue at the time of and/or subsequent to modulation of SERT by the A3R agonist or antagonist. The agonist or antagonist may be introduced into the native brain preparation or tissue more than once. The method may further comprise introduction of a second SERT modulator into the native brain preparation or tissue. In a particular embodiment, the native brain preparation or tissue may be located in a subject, such as a human subject. In such embodiments, agonist may be delivered orally, via subcutaneous, intraspinal or intravenous injection. The antagonist may be administered on a chronic basis. The subject may suffer from depression, an anxiety disorder, obsessive compulsive disorder or autism.

In another embodiment, there is provided a method of desensitizing serotonin transporter (SERT) function in a native brain preparation or tissue comprising chronic treatment of said preparation or tissue with an A3 agonist to enhance SERT activity. The A3 agonist may be IB-MECA, CF101, AB-MECA or N6-2-(4-Aminophenyl)ethyladenosine. The method may further comprise measuring 5-HT uptake in said preparation or tissue prior to introducing said A3 agonist, or further comprise measuring 5-HT uptake in said preparation or tissue at the time of and/or subsequent to introducing said A3 agonist, or further comprise introducing into said preparation or tissue a second A3 agonist. The preparation or tissue may be in a subject, such as a human subject. The agonist may be delivered orally, or via subcutaneous, intraspinal or intravenous injection. The subject may suffer from depression, an anxiety disorder, obsessive compulsive disorder or autism.

It is contemplated that any method or composition described herein can be implemented with respect to any other method or composition described herein. The use of the word, “a” or “an” when used with the term “comprising” in the specification and/or claims may mean “one,” “one or more,” “at least one,” or “one or more than one.” Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C—Effect of A₃ adenosine receptor (A₃AR) agonist IB-MECA on 5-HT uptake in mouse midbrain synaptosomes. (FIG. 1A) Dose response: Synaptosomes were treated with IB-MECA at indicated concentrations for 10 min at 37° C., followed by a 5-min incubation with [³H]-5-HT (final concentration: 20 nM). Non-specific uptake was assessed by incubation of synaptosomes with 10 μM paroxetine for 10 min before adding [³H]-5-HT. (FIG. 1B) Time course of IB-MECA effects on SERT activity. Synaptosomes were incubated with IB-MECA (10 nM) for the indicated times at 37° C. prior to 5-HT uptake assays. (FIG. 1C) Kinetics of IB-MECA stimulation. Synaptosomes were treated with vehicle, or MRS1191 (1 μM) for 10 min followed by IB-MECA (10 nM) for 10 min prior to transport assays. Data were fit to a Michaelis-Menten equation (single site) to derive values for 5-HT Kₘ (vehicle: 0.92+/−0.25 μM, IB-MECA: 0.80+/−0.25 μM; MRS1191+IB-MECA: 0.82+/−0.29 μM) and Vₘₐₓ (vehicle: 1762+/−173; IB-MECA: 2544+/−281**; MRS1191+IB-MECA: 1791+/−218 fmol/mg protein/min) (values are expressed as mean values (n=3)±SEM. **P<0.01 vs. control (Student's t test). Values in FIGS. 1A and 1B are expressed as the mean of five experiments ±SEM. *P<0.05, **P<0.01 vs controls (One-Way ANOVA, Dunnett).

FIGS. 2A-B—Effects of NECA and R-PIA on 5-HT uptake. Synaptosomes from mouse midbrain (C57BL/6) were treated with NECA, a non-specific adenosine receptor agonist (FIG. 2A), or (FIG. 2B) R-PIA (a A₁AR agonist), at the indicated concentrations for 10 min at 37° C. prior to 5-HT uptake assays. Values are expressed as the mean of three experiments ±SEM. **P<0.01 vs. controls (one-way ANOVA, Dunnett).

FIGS. 3A-B—Loss of IB-MECA stimulation of 5-HT uptake in midbrain synaptosomes derived from A₃AR^{-/-} (A₃KO) mice. Synaptosomes from midbrain (FIG. 3A) or hippocampus (FIG. 3B) of either wild-type mouse (wt) or A₃ adenosine receptor knockout mouse (A₃KO) were treated with vehicle (open bars) or IB-MECA (solid bars, 10 nM) for 10 min at 37° C. followed by incubation with [³H]-5-HT (20 nM). The basal 5-HT uptake (fmol/min/mg protein) in midbrain for A₃AR^{+/+} and A₃AR^{-/-} are 1016.7+/−66.3 and 1017.5+/−213.5, respectively; and in hippocampus for A₃AR^{+/+} and A₃AR^{-/-} are 976.1+/−114.4 and 983.8+/−126.1, respectively. Values are expressed as mean (n=4) SEM. **P<0.01 vs. control (Student's t test).

FIGS. 4A-B—Brain region- and neurotransmitter-specific stimulation of IB-MECA. Synaptosomes from mouse striatum (FIG. 4A) and midbrain (FIG. 4B) were treated with vehicle (open bars) or IB-MECA (solid bars) for 10 min prior to assays for 5-HT, DA or GABA uptake. Values are expressed as mean (n=5) SEM. **P<0.01 vs. vehicle control (Student's t test).

FIGS. 5A-D—A₃AR agonist effects on 5-HT uptake are blocked by A₃AR, PKG and p38 MAPK inhibitors. (FIG. 5A) Dose-response of DT-2, a peptide-based PKG inhibitor. Synaptosomes from mouse midbrain were treated with DT-2 at the indicated concentrations for 10 min at 37° C. followed by 5-HT uptake assays. (FIG. 5B) Inhibitors of PKG (H8, 0.1 μM and DT-2, 1.0 μM) and A₃AR (MRS1191, 1.0 M) block IB-MECA stimulated SERT activity. Synaptosomes were incubated with vehicle, H8, DT-2, W45 (10 μM) or MRS1191 for 10 min at 37° C., followed by a 10 min-treatment with IB-MECA (10 nM) prior to 5-HT uptake assay. (FIG. 5C) Dose-response of SB203580, a p38 MAPK inhibitor. Synaptosomes were treated with SB203580 at indicated concentrations for 10 min at 37° C. followed by 5-HT uptake assays. (FIG. 5D) p38 MAPK inhibitor abolishes IB-MECA stimulated 5-HT uptake. Synaptosomes were incubated with vehicle, SB203580 (1.0 μM) or SB202474 (10 μM) for 10 min at 37° C., followed by a 10 min-treatment with IB-MECA (10 nM) prior to 5-HT uptake assay. Values are expressed as the mean of three or more experiments ±SEM. *P<0.05, **p<0.01 vs controls (one-way ANOVA, Dunnett for FIGS. 5A and 5C; two-way ANOVA for FIGS. 5B and 5D).

FIGS. 6A-B—In vivo assessment of IB-MECA effects on 5-HT clearance (FIG. 6A) Effect of IB-MECA on 5-HT clearance in CA3 region of hippocampus of anesthetized rats as monitored by chronoamperometry. 5-HT was locally applied by pressure-ejection until a reproducible signal was obtained. IB-MECA was then locally applied, followed again by 5-HT at 2 and 5 min post-IB-MECA, then at 5 min intervals thereafter until 5-HT clearance parameters returned to pre-drug baseline levels. Data shown are for 20 min following drug administration. IB-MECA (25 μmol) significantly decreased the clearance time for 5-HT. (FIG. 6B) Time course for the effect of IB-MECA (25 μmol) to enhance 5-HT clearance in CA3 region of hippocampus of anesthetized rats. Serotonin was locally applied by pressure-ejection until a reproducible signal was obtained (triplicate replicate signals are averaged and represented by the data point at t=−2 min. At time=0, IB-MECA was locally applied to the hippocampus and then 5-HT was again ejected 2 and 5 min post IB-MECA and at 5 min intervals thereafter until 5-HT clearance parameters returned to pre-drug baseline levels. **P<0.01 vs controls (one-way ANOVA, Dunnett).

### DESCRIPTION OF ILLUSTRATIVE EMBODIMENTS

### I. The Present Invention

A₃ARs are expressed in brain (Zhou et al., 1992; Dixon et al., 1996) and implicated in a variety of conditions including anxiety and depression (Fedorova et al., 2003). Although the inventors have shown A₃AR modulation of SERT in RBL-2H3 cells, a peripheral mast cell line, there is no evidence that A₃AR linked pathways modulate serotonin transport in the brain. The inventors thus sought evidence for A₃AR control of SERT activity in mouse and rat brain preparations, using both in vitro studies of 5-HT uptake in brain synaptosomes and in vivo studies of clearance of pulse-applied 5-HT as assessed by chronoamperometry. Consistent with results in RBL-2H3 cells, the A₃AR specific agonist IB-MECA induces a rapid and significant stimulation of 5-HT uptake in mouse midbrain and hippocampal synaptosome. These effects are blocked by MRS1191, a specific A₃AR antagonist, as well as by PKG and p38 MAPK inhibitors. Moreover, IB-MECA stimulation of SERT is lost in synaptosomes prepared from A₃AR knockout (A₃AR^{-/-}) mice. Finally, chronoamperometry studies reveal a role for A₃AR control of 5-HT clearance, supporting a role for A₃ARs in sustaining serotonergic signaling in vivo, revealing a novel target of potential relevance in 5-HT-linked brain disorders and their amelioration.

Based on these data, the inventors propose to down-regulate SERT activity though a selective mechanism, i.e., by inhibiting A₃AR function. Though applicants are not bound by this particular theory, it is believed that A₃AR play a role in the upregulation of SERT function to a “high” activity level that can be associated with certain pathologic states. Rather than blocking SERT directly, as is done with SSRI's, the inventors seek to block the upregulation of SERT using either A3AR agonists that could induce desensizitization and downregulation of the receptor, or A₃AR antagonists that could attenuate A3AR upregulation of SERT without blocking of the basal function of SERT, which is necessary for many normal processes. Such regulation is difficult to achieve with present SERT inhibitors, such as SSRI's, as they require a transporter occupancy of 85-95%. Therefore, the transporters are essentially totally inactivated at therapeutic doses of the SSRIs. In contrast, the present discovery of the role of A₃AR in SERT stimulation provides an alternative mechanism to more finely regulate SERT function, thereby possibly avoiding the deficiencies associated with current modes of treatment. For example, loss of SERT activity leads to a tonic reduction in neuronal 5-HT levels due to a failure to recycle 5-HT. As shown below, A3KO mice show no reductions in 5-HT indicating that treatment with A3AR drugs will likely not impact basal levels of this important neurochemical. The details of the invention are described in detail below.

### II. Serotonin Transporter (SERT)

Selective antagonism of serotonin (5-hydroxytryptamine, 5-HT) and noradrenaline (NA) transport by antidepressants is a key element to the current understanding of human behavioral disorders (Ashton et al., 1987). The serotoninergic system modulates numerous behavioral and physiological functions and has been associated with control of mood, emotion, sleep and appetite. Synaptic serotonin (SE), also called 5-hydroxytryptamine or 5-HT, concentration is controlled by the serotonin transporter (SERT) which is involved in reuptake of serotonin into the pre-synaptic terminal. In several studies, 5-HT uptake and/or transport sites have been found to be reduced in platelets of patients suffering from depression and reduced in post-mortem brain samples of depressed patients and suicide victims (Meltzer et al., 1981; Suranyi-Cadotte et al., 1985; Briley et al., 1993; Paul et al., 1981; Perry et al., 1983). The cloning of the human SERT protein by Ramamoorthy et al. (1993), shows that human SERT is encoded by a single gene that is localized to chromosome 17q11.1-17q12 and encodes for a 630-amino acid protein. The hSERT is a Na⁺- and Cl⁻ coupled serotonin transporter and has been found to be expressed on human neuronal, platelet, placental, and pulmonary membranes (Ramamoorthy et al., 1993).

The SERT has been associated with depression and anxiety (Soubrie, 1987; Barnes, 1988); obesity (Blundell, 1986; Silverstone et al., 1986); alcoholism (Naranjo et al., 1987); post-anoxic intention myoclonus (Van Woert et al., 1976); acute and chronic pain (Le Bars, 1988); as well as sleep disorders (Koella, 1988). SERT has also been shown to mediate behavioral and/or toxic effects of cocaine and amphetamines (Ramamoorthy et al., 1993). A variety of specific serotonin reuptake inhibitors (SSRIs) such as fluoxetine and paroxetine have been developed for the treatment of depression (reviewed in Schloss, 1998). There has also been an association of SERT with autism (Sutcliffe et al., 2005) and studies linking use of SSRIs for treatment of OCD.

### III. Adenosine Type 3 Receptors

Adenosine regulates a number of physiological functions through cell membrane receptors. Four different adenosine receptors have been identified and classified as A₁, A_{2A}, A_{2B} and A₃. These receptors are members of the G-protein coupled receptor family. However, while A_{2A} and A_{2B} receptors stimulate adenylyl cyclase and increase cAMP levels, A₁ and A₃ receptor subtypes produce the opposite effect. A₁ receptors are usually found on “working” cells and mediate decreases in oxygen demand. Thus, A₁ agonists are useful in the treatment of renal failure, arrhythmias, diabetes Type II, myocardial ischaemia and neurodegenerative disorders. The A₃ receptor, though not yet as well understood, seems to be involved in inflammatory and neurodegenerative diseases, asthma and cardiac ischaemia, but some paradoxical effects have been observed.

Zhou et al. (1992) cloned the A3 adenosine receptor from rat brain. Subsequently, using rat A3R as probe, Sajjadi & Firestein (1993) cloned A3R from a heart cDNA library. This protein had a sequence consistent with other G protein-coupled receptors, including 7 putative transmembrane motifs and 3 potential N-glycosylation sites. The rat A3 receptor protein is about 50 to 60% identical to the A1 and A2 receptors and has been shown to be an inhibitor of adenylate cyclase activity. Human A3R shares about 71% homology with rat A3R. Northern blot analysis detected a transcript of about 2 kb expressed primarily in placenta, lung, heart, liver, and kidney. Little expression was initially detected in skeletal muscle or brain. Salvatore et al. (1993) cloned A3R from a brain striatal cDNA library, showing 72% and 85% overall identity with rat and sheep A3R, respectively. Northern blot analysis detected the 2-kb transcript expressed at highest amounts in lung and liver, at moderate levels in brain and aorta, and at low levels in testis and heart. No expression was detected in spleen or kidney. Murrison et al. (1996) determined that the AR3 gene contains 2 exons separated by a single intron of about 2.2 kb. Importantly, no data presently describes the presence of A3Ars on serotonin neurons or their ability to modulate SERT activity.

### IV. Modulators

A. Modulators of A3 Receptors

A variety of A3R-selective and -specific modulators are known in the art. In particular, U.S. Pat. Nos. 6,358,964, 6,407,236, 6,448,253, 6,673,802, 6,914,053 and 6,921,825, and U.S. Patent Publications 2006/0178385, 2006/0052331, 2006/0040959, 2005/0250729, 2005/0234056, 2005/0119289, 2004/0204481, 2004/0106572, 2004/0121978, 2004/0067932, 2003/0073708, 2003/0144266, 2003/0143282, and 2003/0078232, each of which are hereby incorporated by reference.

B. A3 Agonists

Specific A3 agonists IB-MECA, CF101, AB-MECA and N6-2-(4-Aminophenyl)ethyladenosine. Suitable agonists are disclosed in U.S. Pat. Nos. 7,064,112, 6,586,413, 6,211,165, 5,573,772 and 5,443,836, and U.S. Patent Publications 2008/0051365, 2006/0142237, 2006/0084626, 2004/0106572, 2003/0143282, 2003/0096788 and 2003/0092668, each of which are hereby incorporated by reference.

### V. Depression and Modulation of SERT Activity in CNS

There is a long-standing belief that mood disorders involve disruption of brain 5-HT pathways and that medications that directly target SERT are beneficial in depression, anxiety disorders, OCD and autism (Owens and Nemeroff; 1998). Morever, genetic changes in SERT have been observed to correlate with anxiety traits (Lesch et al., 1996) and risk for depression and suicide (Caspi et al., 2005) as well as autism (Sutcliffe et al., 2005). The present invention therefore seeks to address these and other disease states by providing indirect antagonism of SERT function, more specifically, antagonists of A₃AR or A3 agonists.

Thus, the present invention calls for the administration, to appropriate subjects, of pharmaceutical compositions comprising A₃AR antagonists or A3 agonists. Pharmaceutically acceptable carriers that may be used with the compounds comprise, but not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

For ophthalmic use, the compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions may be formulated in an ointment such as petrolatum.

The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The dose administered to a patient should be sufficient to effect a beneficial response in the subject over time. The dose will be determined by the efficacy of the particular modulators employed and the condition of the subject, as well as the body weight or surface area of the area to be treated. The size of the dose also will be determined by the existence, nature, and extent of any adverse side-effects that accompany the administration of a particular compound or vector in a particular subject. In determining the effective amount of the compound to be administered, a physician may evaluate circulating plasma levels of the compound, compound toxicities, and the production of anti-compound antibodies. In general, the dose equivalent of a compound is from about 1 ng/kg to 10 mg/kg for a typical subject. Administration can be accomplished via single or divided doses.

### VI. Screening Methods

In particular embodiments, the present invention provides methods for high throughput screening for A3R-targeted modulators of the serotonin transporter. Such assays will typically comprise the use of whole cells or brain tissue preparations derived from experimental animals where SERT is expressed and transport may be directly or indirectly measured, as discussed below. Alternatively, SERT and A3ARs can be coexpressed in a transfected model cell system.

A. Measurement of Transport

In some embodiments, the present invention provides a novel and rapid method for analysis of transport by a serotonin transporter that comprises the measurement of uptake and/or accumulation of serotonin and analogues thereof that are specifically taken up by the transporter. Typically, this is accomplished by measuring the uptake or binding of radiolabeled serotonin (e.g., [3H]serotonin) or a radiolabeled antagonist such as [³H]citalopram, [³H]paroxetine, or [¹²⁵I]RTI-55. Conventional assays involves the uptake of radiolabeled 5-HT where antagonist sensitivity is measured for inhibition of serotonin accumulation or the inhibition of labeled antagonist binding to intact cells expressing SERT or to membranes from intact cells expressing SERT. Basically, cells transfected with a SERT construct are washed in assay buffer followed by a preincubation in 37° C. assay buffer containing 1.8 g/L glucose. This is followed by an incubation period, about 10 min, at 37° C. in the presence of [³H]-5-HT, or a radiolabeled antagonist such as [³H]citalopram, [³H]paroxetine, or [¹²⁵I]RTI-55. Details of this assay are provided in the Examples.

1. Scintillation Proximity Assays

Measurement of transport may also be involve scintillation proximity assays, which is used to count the accumulated radiolabel on plates having scintillant embedded in them. Basically, cells are plated at 50% confluence on 0.4-μm pore size 6.5-mm Transwell cell culture filter inserts and grown for 7 days. A cell monolayer growing on the porous membrane of the cell culture filter insert effectively separates each well in the cell culture plate into two chambers. The apical membranes of epithelial cells plated on these filters faces the chamber above the cells and the basolateral membranes face the lower chamber through the filter. After one wash each of the apical (upper chamber) and basolateral (lower chamber) sides of the monolayer with PBS/Ca/Mg, the cells are incubated in PBS/Ca/Mg containing ³H-labeled substrate either in the upper or the lower chamber at 22° C. At the end of the incubation cells are washed either three times from the apical side and once from the basolateral side (when ³H-labeled substrate was present in the upper chamber) or once from the apical side and three times from the basolateral side (when substrate was present in the lower chamber). The apical side of the cells are washed by adding 0.2 ml of ice-cold PBS to the upper chamber and aspirating. The basolateral side of the cells are washed by pipetting ice-cold PBS over the bottoms of the filter inserts. After the washes, the filters with cells attached are excised from the insert cups, submerged in 3 ml of Optifluor scintillation fluid (Packard Instrument Co., Downers Grove, Ill.), and counted in a Beckman LS-3801 liquid scintillation counter. Transport assays on 48-well plates were described previously (Gu et al., 1994).

2. Voltage and Patch Clamp

The present invention also employs a means of determining the serotonin transporter activity or function by measuring the change in movement across a membrane, when the transporter is active. This may be accomplished using the voltage clamp technique, as is well known in the art, this allows the gating properties of the voltage-gated channels to be analyzed.

In short, the voltage clamp technique is a procedure whereby the transmembrane voltage of a membrane segment is rapidly set and maintained at a desired level. Once the membrane potential is controlled, the current flowing through the channels in that segment can be measured.

The patch clamp technique allows the voltage clamp technique to be applied to a small patch of membrane containing a single voltage-sensitive channel. The basic idea behind a patch clamp experiment is to isolate a patch of membrane so small that it contains a single voltage-gated channel. Once this patch of membrane is isolated, the single channel can be voltage clamped. Using this technique, the gating properties of the serotonin transporter can be characterized.

B. Other Methods of Measurement of Transport

Other methods of measurement contemplated in the present invention may involve fluorescence microscopy. This may involve the use of fluorescent substrates, some of which are contemplated to be analogs of other native neurotransmitters.

1. Microscopy

Fluorescent microscopy is used to measure transport using serotonin or analogues thereof which are fluorescent substrates for the serotonin transporter. Cells that either endogenously or exogenously express a serotonin transporter are isolated and plated on glass bottom Petri-dishes or multi-well plates that may typically be coated with poly-L-lysine or any other cell adhesive agent. Cells are typically cultured for three or more days. The culture medium is then aspirated and the cells are mounted on a Zeiss 410 confocal microscope. During the confocal measurement cells remain without buffer for approximately thirty seconds. Background autofluorescence is established by collecting images for ten seconds prior to the addition of the buffer and serotonin or analogues thereof. As serotonin or an analogue thereof has a large Stoke shift between excitation (lₘₐₓ=488 mn) and emission maxima (lₘₐₓ=610 nm), the argon laser is tuned to 488 nm and the emitted light filtered with a 580-630 nm band pass filter (lₘₐₓ=610 nm). The substantial red shift can be exploited to reduce background auto-fluorescence produced in the absence of substrate. The gain (contrast) and offset (brightness) for the photomultiplier tube (PMT) may be set to avoid detector saturation at the higher serotonin concentrations that may be used in certain experiments. The effects of photo-bleaching on serotonin accumulation may also be determined by examining the rate of serotonin accumulation and decay at various acquisition rates. In a constant pool of serotonin, rates as high as 20 Hz (50 msec/image) can be set.

2. Fluorescence Anisotropy Measurements

To evaluate serotonin or analogues thereof binding to the surface membranes, cells expressing a serotonin transporter may be exposed to serotonin or analogues thereof with horizontal polarizer, with the polarizer rapidly switching to the vertical position. Cells may be imaged with alternating polarizations for 3 min to measure light intensity in the horizontal (Iₕ) and vertical (Iᵥ) positions in order to calculate the anisotropy ratio, r=(Iᵥ−gIₕ)/(Iᵥ+2 g Iₕ). The factor g may be determined by using a half wave plate as described by Blackman et al. (1996). In this formulation, r=0.4 implies an immobile light source. Surface anisotropy can be measured at the cell circumference over 1 pixel width (0.625 mm). Cytosolic anisotropy can be measured near the center of the cell, approximately 5 pixel widths from the membrane.

3. Image Analysis

The fluorescent images may be processed using suitable software. For example, fluorescent images may be processed using MetaMorph imaging software (Universal Imaging Corporation, Downington Pa.). Fluorescent accumulation may be established by measuring the average pixel intensity of time resolved fluorescent images within a specified region identified by the DIC image. Average pixel intensity is used to normalize among cells.

4. Single Cell Fluorescence Microscopy

In some embodiments, the invention provides measurement of transporter characteristics at the single-cell level. Single-cell fluorescence microscopy provides a powerful assay to study rapid serotonin uptake kinetics from single cells.

5. Automation

The inventors further contemplate that all these methods are adaptable to high-throughput formats using robotic fluid dispensers, multi-well formats and fluorescent plate readers for the identification of serotonin transport modulators.

C. In vivo Microdialysis

Microdialysis may be used in the present invention to monitor interstitial fluid in various body organs with respect to local metabolic changes. This technique may also be experimentally applied in humans for measurements in adipose tissue. In the present invention, the release of serotonin in the mouse brain, in response to stimuli may be analyzed using this technique.

Microdialysis procedure involves the insertion through the guide cannula of a thin, needle-like perfusable probe (CMA/12.3 mm×0.5 mm) to a depth of 3 mm in striatum beyond the end of the guide. The probe is connected beforehand with tubing to a microinjection pump (CMA-/100). The probe may be perfused at 2 ml/min with Ringer's buffer (NaCl 147 mM; KCl 3.0 mM; CaCl₂ 1.2 mM; MgCl₂ 1.0 mM) containing 5.5 mM glucose, 0.2 mM L-ascorbate, and 1 mM neostigmine bromide at pH 7.4). To achieve stable baseline readings, microdialysis may be allowed to proceed for 90 min prior to the collection of fractions. Fractions (20 ml) may be obtained at 10 minute intervals over a 3 hour period using a refrigerated collector (CMA170 or 200). Baseline fractions may be collected, following the drug or combination of drugs to be tested, been administered to the animal. Upon completion of the collection, each mouse may be autopsied to determine accuracy of probe placement.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Reagents. N6-(3-iodobenzyl)-N-methyl-5′carbamoyladenosine (IB-MECA), 5′-N-ethyl-carboxamidoadenosine (NECA), N-[2-(methylamino)ethy]-5-isoquinoline-sulfonamide (H8), (R)—N⁶-phenylisopropyladenosine (R-PIA), 3-ethyl-5-benzyl-2-methyl-phenylethynyl-6-phenyl-1,4(±)dihydropyridine-3,5-dicarboxylate (MRS1191) were purchased from Sigma (St. Louis, Mo.); SB203580 was obtained from Alexis Biochemicals (San Diego, Calif.). DT-2 was a kind gift from Dr. Wolfgang Dostmann, U. Vermont (Taylor et al., 2004). [³H]5-HT (5-hydroxy[³H]tryptamine trifluoroacetate, 107 ci/mmol) was purchased from Amersham Biosciences Inc, (Piscataway, N.J.). All other biochemical reagents were of the highest grade possible and obtained from Sigma (St Louis, Mo.). A₃AR^{-/-} mice (Salvatore et al., 2000), generously provided by Dr. Marlene Jacobson (Merck, West Point, Pa.), were maintained on a C57BL/6 background and were and housed and bred in the Vanderbilt University Vivarium with water and food provided ad libitum.

Synaptosomal studies. C57BL/6 mice (Harlan Sprague Dawley, Inc., Indianapolis, Ind., A₃AR^{+/+}) and A₃AR^{-/-} mice were used in these studies following an approved protocol of the Vanderbilt University Institutional Animal Care Use Committee. Synaptosomes were prepared as described previously (Zhu et al., 2005). In brief, mouse midbrain, hippocampus, and striatum were dissected on ice after sacrifice, homogenized in 0.32 M D-glucose at 400 rpm using a Teflon-glass tissue homogenizer (Wheaton Instruments, Millville, N.J.). The homogenized tissue was centrifuged at 800 xg for 10 min at 4° C. The supernatant was transferred to new centrifuge tubes and centrifuged at 10,000×g for 15 min at 4° C. Synaptosomal pellets were re-suspended in Krebs-Ringer's HEPES (KRH) buffer containing 130 mM NaCl, 1.3 mM KCl, 2.2 mM CaCl₂, 1.2 mM MgSO₄, 1.2 mM KH₂PO₄, 1.8 g/L D-glucose, 10 mM HEPES, pH 7.4, 100 μM pargyline, and 100 μM ascorbic acid. Synaptosomal suspensions were assessed for protein content (Bio-Rad, Hercules, Calif.) and maintained on ice until dispensed for 5-HT transport assays.

5-HT Transport Assays. [³H]5-HT transport activity was assayed as described previously (Zhu et al., 2006). Briefly, 30-50 μg synaptosomes per sample (in a total volume of 200 μL) were pre-incubated at 37° C. in a shaking water bath for 10 min. Vehicle or modifiers were then added for 10-20 min. After a 5 min incubation with [³H]5-HT (20 nM), [³H]DA (Dihydroxyphenylethylamine, 3,4-[7-3H], PerkinElmer, Boston, Mass.; 50 nM), or[3H]GABA (γ-[2, 3-3H(N)]-Aminobutyric Acid, PerkinElmer, Boston, Mass.; 50 nM) at 37° C., uptake was terminated by filtration through (PEI-coated) GF/B Whatman filters using a Brandel Cell Harvester (Brandel, Gaithersburg, Md.). Filters were washed three times with ice-cold KRH buffer, immersed in scintillation liquid for 8 hr, and radioactivity accumulated quantitated by scintillation spectrometry (Beckman, Fullerton, Calif.). Counts obtained from the filtered samples were corrected for non-specific uptake using parallel samples incubated at 37° C. with paroxetine (1 μM, for SERT) or GBR 12935 (1 μM, for dopamine transporter) or when incubated on ice (GABA).

Assays for 5-HT level from whole tissue of the brain regions. To assess levels of 5-HT and 5-HIAA in dissected brain regions (hippocampus, midbrain, striatum and frontal cortex), tissues were dissected on ice and quickly frozen in liquid nitrogen and stored at −80° C. until HPLC analysis. On the day of analysis, brain regions were homogenized in 100-750 μl of 0.1M TCA, containing 10⁻² M sodium acetate, 10⁻⁴ M EDTA and 10.5% methanol (pH 3.8). Samples were centrifuged at 10000×g for 20 min. Supernatants were stored at ⁻80° C. and pellets processed for protein analysis (Bio-Rad, Hercules, Calif.). Supernatants were analyzed for biogenic monoamine levels by HPLC (Phenomenex Nucleosil, 5u, 100A) C18 HPLC column (150×4.60 mm) using an Antec Decade II (oxidation: 0.5) electrochemical detector in the Center for Molecular Neuroscience Neurochemistry Core, Raymond Johnson Core Manager. Data were converted into picomoles per milligram protein, with comparisons between wild type and A₃AR^{-/-} samples performed using a Student's unpaired t test.

In vivo Chronoamperometry Studies. Male Sprague-Dawley rats (Harlan, Indianapolis, Ind.), weighing 280-380 g, were used for all chronoamperometry experiments, as approved by the UT San Antonio Health Sciences Center Institutional Animal Care and Use Committee and were in strict accordance with the NIH Guide for the Care and Use of Laboratory Animals. Rats were anesthetized by intraperitoneal injection of chloralose (85 mg/kg) and urethane (850 mg/kg). A tube was inserted into the trachea to facilitate breathing, and the animal was placed into a stereotaxic frame. The electrode micropipette recording assembly was lowered into the CA3 region of the dorsal hippocampus (AP, −4.1; ML, +3.5; DV, −3.6) (Paxinos and Watson, 1986). Body temperature was maintained at 37±1° C. by a water-circulated heating pad (Seabrook, Cincinnati, Ohio, U.S.A.). High-speed chronoamperometric recordings were made using the FAST-12 system (Quanteon, Lexington, Ky.), as described previously (Daws et al., 2000). In brief, carbon fiber electrodes (tip diameter, 30 μm; Quanteon) were coated with Nafion (5% solution; Aldrich Chemical Co., Milwaukee, Wis.), to prevent interference from anionic substances in the ECF. Electrodes were tested for sensitivity to the 5-HT metabolite, 5-hydroxyindoleacetic acid (5-HIAA; 250 μM), and calibrated with six increasing concentrations of 5-HT ranging from 0.5 to 3.0 μM. Only electrodes displaying a selectivity ratio for 5-HT over 5-HIAA of >1,000:1 and a linear response (r²≧0.997) to 5-HT (0.5-3.0 μM) were used. The detection limit for the measurement of 5-HT levels was defined as the concentration that produced a response with a signal-to-noise ratio of 3. The electrochemical recording assembly consisted of a Nafion-coated, single carbon fiber electrode attached to a four- or seven-barreled micropipette. The assembly was constructed such that the electrode and micropipette tips were separated by 300±20 μm. The tip diameter of each barrel of the multibarelled micropipette was between 10 and 15 μm. Barrels were filled with 5-HT (200 μM), the A₃AR agonist IB-MECA (200, 400 or 800 nM), or vehicle. All drugs were dissolved in 0.1 M phosphate-buffered saline (PBS) with 100 μM ascorbic acid added as an antioxidant. 5-HT was pressure-ejected (5-25 psi for 0.25-3 s) at 3-10-min intervals until a reproducible signal was obtained (usually three or four applications). The mean±SEM number of picomoles of 5-HT delivered was 10±1 in a volume of 50±3 nl as measured by determining the amount of fluid displaced from the micropipette using a dissection microscope fitted with an eyepiece reticule. Once the 5-HT signal was reproducible, vehicle or drug was applied 60-90 s before the next application of 5-HT to allow sufficient time for diffusion of drug to areas around the recording electrode. These solutions were pressure-ejected over 10-20 sec to minimize disturbances to the baseline electrochemical signal. IB-MECA was ejected in a volume of 125 nl to deliver 25, 50 or 100 μmol. An equivalent volume of vehicle was ejected as a control.

Statistical Analyses. All data derive from experiments replicated a minimum of 3 times. Statistical analyses, comparing baseline and compound-modified uptake, were performed with GraphPad Prism (GraphPad, San Diego, Calif.) using One- and Two-Way Analyses of Variance (ANOVA) with subsequent planned comparisons (Dunnett, Bonferroni), as well as Student t tests, as noted in the Legends. P<0.05 was taken as significant for all evaluations.

### Example 2

### Results

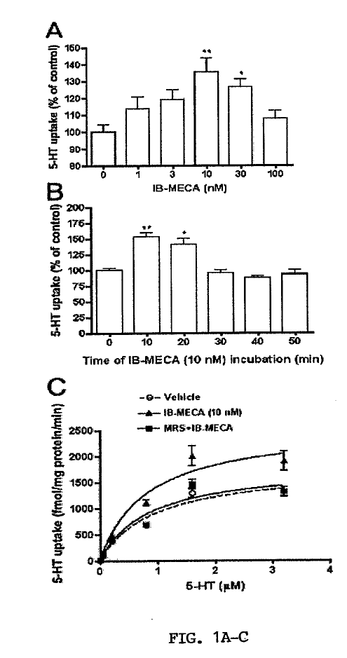

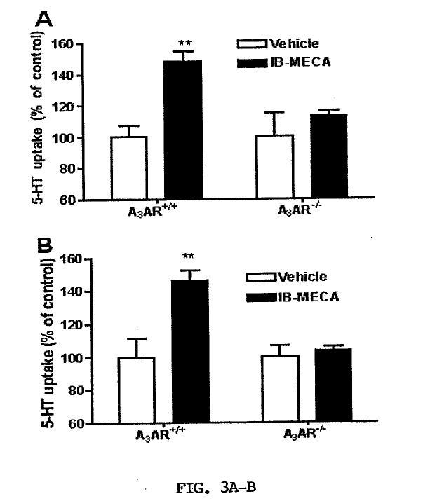

Activation of A₃ARs in mouse brain synaptosomes induces an increase in 5-HT uptake. N⁶-(3-Iodobenzyl)adenosine-5′-N-methyluronamide (IB-MECA) (Jacobson et al., 1993; Gallo-Rodriguez et al., 1994) was used to examine A₃AR-dependent regulation of SERT in mouse midbrain synaptosomes. IB-MECA exerted a concentration- and time-dependent stimulation of 5-HT transport activity (FIGS. 1A and 1B). IB-MECA pretreatment of synaptosomes for 10 min resulted in stimulation of 5-HT transport that peaked at 10 nM (130-150% of control levels, FIG. 1A). Higher concentrations of IB-MECA did not further increase 5-HT transport; rather, reduced efficacy was apparent above 10 nM and when exceeding 100 μM, IB-MECA even induced a decrease in 5-HT uptake (data not shown), effects not pursued in the present study. Using a concentration of 10 nM IB-MECA, the inventors examined the time course of IB-MECA stimulation, where effects were observed to reach a maximum at 10-20 min post-treatment with less efficacious actions evident with longer treatments (FIG. 1B). A similar action was observed for IB-MECA in hippocampal synaptosomes (see below, FIG. 3). Surprisingly, this action mirrors the activity of A3AR ligands in RBL-2H3 cells (Zhu et al., 2004a), IB-MECA treatments of midbrain synaptosomes significantly increased the SERT Vmax for 5-HT (FIG. 1C). The inventors could detect no significant IB-MECA induced change in 5-HT Km (FIG. 1C). Importantly, MRS1191, a selective antagonist for A₃AR, prevented the stimulatory actions of IB-MECA on 5-HT uptake (FIGS. 1C and 3B). MRS1191 alone did not influence basal 5-HT transport in synaptosomes over a wide range of concentrations (0.01-100 μM, data not shown).

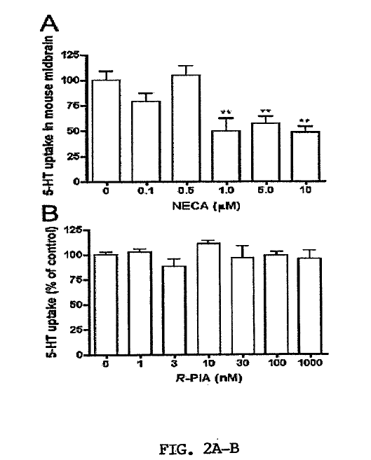

NECA and R-PIA do not stimulate synaptosomal 5-HT uptake. Multiple ARs are present in brain and thus the inventors wished to explore whether the actions of IB-MECA reflect a specific association of the A₃AR with neuronal SERT regulation. NECA is a non-selective AR agonist with a similar potency at A₁, A₂ and A₃ ARs (Similar Ki for rat and mouse A₁, A_{2A} and A₃=6.3, 10 and 113 nM, respectively, with values 3.5-26 fold higher in human and guinea-pig preparations (Feoktistov and Biaggioni, 1997; Maemoto et al., 1997). Whereas NECA stimulates SERT activity in RBL-2H3 cells (Miller and Hoffman, 1994; Zhu et al., 2004a), this agent fails to stimulate SERT activity in midbrain synaptosomes (FIG. 2A). At concentrations above 1 μM, NECA actually inhibited 5-HT transport, suggesting an underlying receptor complexity that may limit conclusions from neural tissues (as compared to cell lines) with nonselective AR agonists. Additionally, the inventors previously found that treatment of SERT/A₁AR co-transfected CHO cells with the A₁AR-specific agonist R-PIA (Zhu et al., 2004a) stimulated SERT activity. Since A₁ARs are abundantly expressed in brain (Yaar et al., 2005) and utilize similar signaling pathways as A₃ARs (Feoktistov and Biaggioni, 1997; Fredholm et al., 2005), the inventors tested the activity of R-PIA for SERT regulation in midbrain synaptosomes. No impact of R-PIA on 5-HT transport was observed across a wide range of concentrations (1˜1000 nM) (FIG. 2B).

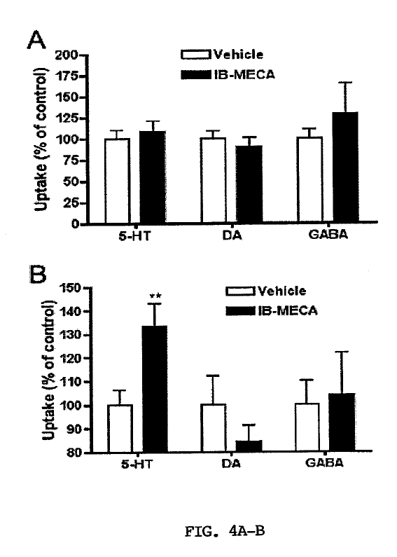

Specificity of IB-MECA stimulation of synaptosomal SERT activity. To verify that MRS1191-sensitive IB-MECA stimulation of synaptosomal SERT activity is mediated by A₃ARs, the inventors compared modulation of 5-HT uptake in midbrain and hippocampal synaptosomes prepared from A₃AR−/− mice (Salvatore et al., 2000) versus wild-type (A₃AR^{+/+}) mice of the same genetic background (FIG. 3). As expected, IB-MECA induced a significant increase (40-50%) in 5-HT transport with A₃AR^{+/+} mice. In contrast, IB-MECA failed to stimulate SERT activity in A₃A^{-/-} mice. Although SERT regulation by IB-MECA was lost in A3AR^{-/-} preparations, basal SERT activity was equivalent in A₃AR^{+/+} and ^{-/-} synaptosomes (FIG. 3, legend), as were tissue 5-HT and 5-HIAA levels (Table 1). Additionally, the inventors explored the regional and substrate specificity of IB-MECA stimulation of SERT activity (FIGS. 4A-D). Unlike midbrain, hippocampal (FIGS. 1-4D) and cortical synaptosomes (data not shown), striatal synaptosomes displayed IB-MECA-insensitive SERT activity (FIG. 4A). In striatal and midbrain preparations, the inventors also assessed IB-MECA effects on DA and GABA transport activity. In assays where clear upregulation of SERT was evident, the inventors observed no stimulation for either DA or GABA uptake. These studies indicate that A₃ARs are not required to maintain 5-HT uptake or 5-HT/5HIAA levels but are essential for the actions of IB-MECA in stimulation of midbrain and hippocampal SERT in vitro.

TABLE 1

| Brain | Genotypes | | | |
|---|---|---|---|---|
| Regions | (A₃AR) | 5-HT | 5-HIAA | 5-HT/5-IAA |
| Frontal | +/+ | 5.34 ± 1.31 | 6.59 ± 4.55 | 1.49 ± 1.49 |
| cortex | −/− | 5.72 ± 0.80 | 12.11 ± 8.36 | 0.70 ± 0.43 |
| Hippo- | +/+ | 5.43 ± 1.73 | 8.96 ± 6.83 | 0.95 ± 0.55 |
| capmus | −/− | 5.84 ± 1.46 | 8.82 ± 4.42 | 0.78 ± 0.28 |
| Midbrain | +/+ | 8.39 ± 0.98 | 13.84 ± 7.93 | 0.80 ± 0.42 |
| | −/− | 9.29 ± 1.76 | 14.98 ± 6.00 | 0.66 ± 0.17 |
| Striatum | +/+ | 6.19 ± 2.72 | 8.50 ± 5.68 | 0.87 ± 0.29 |
| | −/− | 6.41 ± 1.71 | 9.21 ± 4.28 | 0.78 ± 0.22 |
| Brain | | | | |
| Regions | Genotypes | DA | DOPAC | DA/DOPAC |
| Frontal | +/+ | 13.51 ± 9.05 | 4.18 ± 2.96 | 3.38 ± 1.13 |
| cortex | −/− | 11.17 ± 4.88 | 2.93 ± 1.49 | 3.92 ± 0.74 |
| Hippo- | +/+ | 0.37 ± 0.18 | 0.10 ± 0.11 | 3.70 ± 1.53 |
| capmus | −/− | 0.36 ± 0.09 | 0.18 ± 0.13 | 2.00 ± 0.63 |
| Midbrain | +/+ | 1.28 ± 0.22 | 0.70 ± 0.14 | 1.86 ± 0.30 |
| | −/− | 1.39 ± 0.37 | 0.72 ± 0.09 | 1.90 ± 0.39 |
| Striatum | +/+ | 83.47 ± 41.42 | 8.78 ± 5.39 | 10.27 ± 2.26 |
| | −/− | 91.60 ± 43.07 | 9.63 ± 2.50 | 9.05 ± 3.81 |

| Brain Regions | Genotypes | NE | E |
|---|---|---|---|
| Frontal | +/+ | 5.34 ± 1.03 | 0.14 ± 0.11 |
| cortex | −/− | 5.07 ± 0.64 | 0.19 ± 0.14 |
| Hippo- | +/+ | 5.04 ± 1.83 | 0.11 ± 0.06 |
| capmus | −/− | 4.63 ± 1.57 | 0.20 ± 0.17 |
| Midbrain | +/+ | 7.88 ± 2.24 | 0.08 ± 0.08 |
| | −/− | 8.76 ± 3.29 | 0.32 ± 0.25 |
| Striatum | +/+ | 2.80 ± 0.66 | 0.13 ± 0.13 |
| | −/− | 2.02 ± 0.57 | 0.11 ± 0.08 |

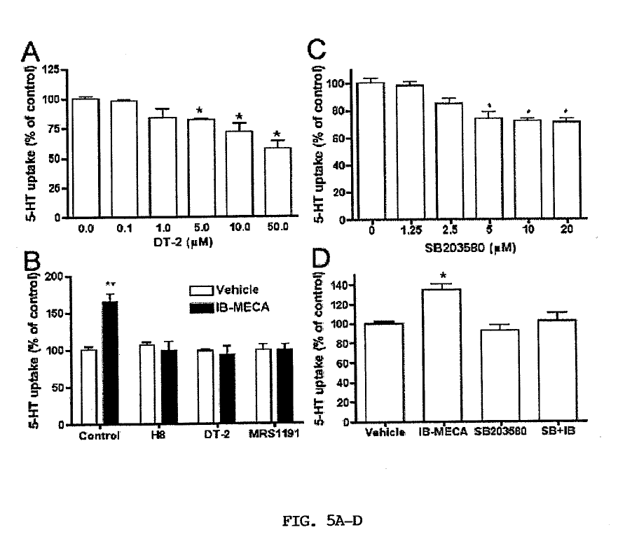

A₃AR stimulation of synaptosomal SERT involves PKG and p38 MAPK signaling pathways. A₃AR stimulation of SERT arises via activation of guanyl cyclase, production of cGMP, and activation of PKG, leading to insertion of intracellular SERT proteins into the plasma membrane (Miller and Hoffman, 1994; Zhu et al., 2004a). Additionally, PKG activation triggers phosphorylation and activation of p38 MAPK, leading to catalytic activation of surface SERTs (Zhu et al., 2004a). To explore whether the same pathways are involved in the synaptosomal actions of IB-MECA reported above, the inventors asked whether PKG and p38 MAPK antagonists attenuate basal and IB-MECA-stimulated SERT activity in midbrain synaptosomes. DT-2, a membrane permeant peptide that potently (Kᵢ=12.5 nM; Dostmann et al., 2000) and selectively inhibits PKG1, fails to impact basal 5-HT uptake until concentrations reach or exceed 5 μM (FIG. 5A). Similarly, the p38 MAPK inhibitor SB203580 attenuated basal SERT activity at concentrations of or above 5 μM (FIG. 5B). When tested at concentrations lacking actions on basal SERT activity, both DT-2 (FIG. 5C) and SB203580 (FIG. 5D) blocked the effects of IB-MECA in stimulation of SERT activity. The nonpeptide PKG inhibitor H8 (0.1 μM), when tested at concentrations lacking basal actions, also blocked IB-MECA regulation of SERT. Together, these findings support a role for PKG and p38 MAPK-linked pathways in the acute modulation of SERT by A₃ARs.

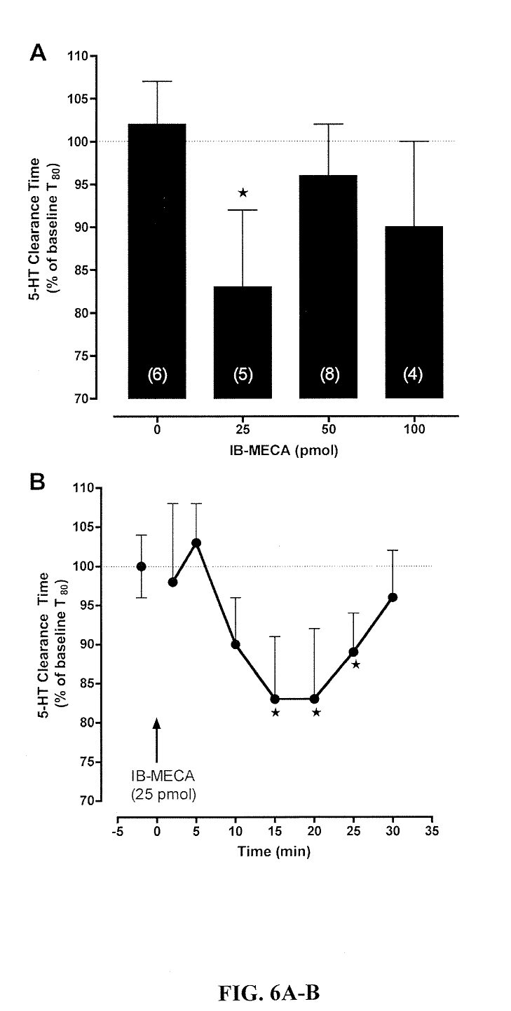

A₃AR-modulate SERT-mediated 5-HT clearance in vivo. In order to evaluate whether A₃ARs can regulate SERTs in vivo, the inventors monitored SERT-mediated 5-HT clearance using high speed chronoamperometry (Daws and Toney, 2006). Chronoamperometric recordings of the clearance rates of exogenously applied 5-HT were obtained from the CA3 region of hippocampus in anesthetized rats. After obtaining a stable baseline for the clearance of injected 5-HT, the inventors injected varying doses of IB-MECA, followed by reapplications of 5-HT. The time to clear 5-HT by 80% (T80) was quantified and plotted as a function of IB-MECA concentration (FIG. 6A) and time (FIG. 6B). Injection of vehicle was without effect across the recording time course. In contrast, the inventors observed a significant reduction in T80 (enhanced clearance rate) with IB-MECA, effects that were both dose- and time-dependent. Low (25 μmol) but not high (50-100 μmol) amounts of IB-MECA injected significantly enhanced 5-HT clearance rate when assessed at 20 min (FIG. 6A). As observed in vitro, the effect of IB-MECA (25 μmol) to enhance 5-HT clearance was not immediate, but rather displayed a time-dependent course with a peak effect at 15-20 min followed by reversal to control levels (FIG. 6B). These findings are the first to support the ability of A3ARs to modulate

SERT in vivo, and are consistent with the inventors' own mouse synaptosomal studies (above).

### Example 3

### Discussion

Adenosine receptors (ARs; A₁, A_{2A}, A_{2B} and A₃) are widely distributed throughout the brain and periphery (Fredholm et al., 2001), and have been implicated in a variety of physiological and pathological conditions, including modulation of neural signaling (Okada et al., 1999; Albasanz et al., 2002), neuroprotection, cardiovascular functions, drug addiction, Parkinson's Disease, Schizophrenia, anxiety, depression and pain (Fredholm, 2003; Halldner et al., 2004). In the periphery, A₃AR activation is cardioprotective (Parsons et al., 2000) and induces an increase in histamine or TNF-α release in rodents (Ramkumar et al., 1993; Salvatore et al., 2000). Deletion of A₃AR in mice enhances adenosine-stimulated coronary blood flow (Talukder et al., 2002) and induces alterations in anxiety/depression-linked behaviors (Fedorova et al., 2003).

In the inventors' previous study, they observed that A₃AR activation stimulates 5-HT uptake in RBL-2H3 cells via a PKG- and p38 MAPK-linked pathway (Zhu et al., 2004a). Although they (Zhu et al., 2004a; Zhu et al., 2005) and others (Samuvel et al., 2005) have established that PKG and p38 pathways regulate SERT in brain preparations, specific receptors linked to these pathways are ill-defined. Recently (Zhu et al., 2006), they demonstrated that IL-1β receptors couple via the p38 MAPK pathway to activate SERT. In the current study, the inventors demonstrated that A₃ARs in the brain constitute a second mechanism for the modulation of CNS SERT activity. The actions of the A₃AR agonist IB-MECA to stimulate 5-HT transport occur across a narrow concentration and time window (10-30 nM for 20 min). This may reflect regulation of SERT by the multiple AR subtypes types found in the brain (Fredholm et al., 2005), with temporal profiles of SERT activity shaped by the particular coupling kinetics of A₃ARs. Although IB-MECA is relatively potent and specific for the A₃AR (K_{D}=1.1 mM), at high concentration, it can also activate A₁ and A₂A ARs (K_{D}˜50 nM in rat and mouse) (Feoktistov and Biaggioni, 1997; Maemoto et al., 1997). Simultaneous stimulation of multiple AR types in the brain could compromise detection of A₃AR-mediated effects on SERT activity. Possibly such issues relate to the inventors' finding that the non-selective AR agonist NECA fails to stimulate 5-HT transport in synaptosomes whereas it stimulates SERT in cultured RBL-2H3 cells. Additionally, ARs exert their action on a variety of neurotransmitter systems (Fredholm et al., 2005), and one AR subtype can also impact the activity of other AR subtypes (Dunwiddie et al., 1997; Okada et al., 1999). The inventors did not observe an ability of the A₁AR selective agonist R-PIA to stimulate 5-HT transport in brain whereas the inventors have found that co-transfected A₁AR can enhance 5-HT uptake via SERT in cotransfected cells (Zhu et al., 2004a). Overall, these findings indicate a selective relationship between A₃ARs and SERT, one that may derive from their co-expression on serotonergic terminals. Unlike SERT where gene expression is limited in the CNS to raphe neurons, gene expression for A₃ARs is widespread throughout the CNS including hippocampus and midbrain (Dixon et al., 1996). Colocalization studies with antibodies specific for SERT and A₃ARs are needed to more finely establish the spatial relationships of these proteins. The inventors also demonstrate that A₃AR-mediated 5-HT transport enhancement can be blocked by H8/DT-2 (general and specific inhibitors of PKG) or SB203580 (a specific p38 MAPK inhibitor), consistent with the participation of both PKG and p38 MAPK in rapid enhancements of SERT activity. As the kinetic studies of A₃AR-mediated stimulation of SERT activity in midbrain synaptosomes demonstrate an increase of Vmax, enhanced trafficking of SERT proteins as demonstrated in RBL-2H3 cells is likely (Zhu et al., 2004a; Zhu et al., 2006). SERT has been identified on both plasma membranes and intracellular vesicles of serotonergic terminals (Pickel and Chan, 1999; Miner et al., 2000; Huang and Pickel, 2002). The latter compartment may be mobilized by A₃AR-triggered PKG activation. Alternatively, SERT recycling rates may be accelerated to permit an increase in transporter surface expression (Loder and Melikian, 2003).

To further validate the A₃AR targeted actions of IB-MECA on SERT, the inventors took advantage of A₃AR^{-/-} mice (Salvatore et al., 2000). Although basal SERT activity, as well as 5-HT and 5-HIAA levels, are normal in A₃AR^{-/-} mice, IB-MECA stimulation of SERT was abolished. Previous behavioral studies with A₃A^{-/-} mice demonstrate an increase in locomotor behavior in the open field test, an increase in open arm entries on the elevated plus maze test, and increased transitions in the light/dark box test (Fedorova et al., 2003), suggesting a reduction in the level of anxiety or fearfulness. Additionally, A₃AR impacts immobility in the Porsolt swim test and the tail suspension test, two behaviors typically offset by acute SSRI administration (Fedorova et al., 2003). Although the inherent limitations of knockout studies preclude more definitive conclusions, these findings lend support to a functional relationship between A₃AR activation, SERT activity and 5-HT signaling. Possibly, the loss of A₃ARs may prevent environment-triggered increases in SERT activity that could be anxiogenic and over time such deficits result in increased exploratory behavior and diminished fearfulness. At times when environmental stress should be translated into behavioral activation, failure to activate SERT may prevent normal struggling responses in swim and tail suspension tests. Temporally-controlled A₃AR gene ablation or knockdown restricted to raphe neurons (Scott et al., 2005) should be helpful to test such ideas.

The actions of IB-MECA in vitro appear relatively selective for SERT as neither DA nor GABA transport were modulated in the same preparations where 5-HT uptake was increased. These findings indicate that SERT modulation does not arise from a nonspecific action of IB-MECA on ion gradients or membrane potential that commonly support the transport of each of these substrates and are consistent with the inventors' implication of PKG and p38 MAPK pathways in SERT modulation. Perhaps more interestingly, these results show that IB-MECA-induced SERT modulation is brain region-dependent, suggesting possible differences in A₃AR abundance, localization or coupling as related to SERT expression. Alternatively, negative regulatory pathways unique to striatum or the presence of limited regulatory capacity may preclude detection of an ability of A₃ARs to modulate SERT in this region. Striatal SERTs participate in actions of 5-HT in motor and reward pathways whereas hippocampal and cortical SERTs constrain the actions of 5-HT in cognitive and mood circuits. The inventors' findings of region-specific control of SERTs by A₃ARs may thus prove important for the development of medications that modify some, but not all facets of 5-HT signaling. Clearly, extension of these findings for medication development requires additional in vivo studies. Important validation of a role of A₃AR activation in regulation of SERT activity in vivo arises from the inventors' chronoamperometry studies, where they assessed the clearance of pulse-applied 5-HT. Specifically, they observed a dose and time-dependent effect of IB-MECA administration to increase 5-HT clearance, consistent with the stimulation of SERT activity observed in synaptosomes. The present findings are also consistent with a prior in vivo dialysis study that reported an A₃AR-dependent reduction in extracellular 5-HT (Okada et al., 1999). These authors, however, did not identify the mechanism by which A₃ARs reduce 5-HT, nor whether SERT was involved.

The results from the current study also raises the question as to the source of endogenous adenosine that acts through A₃AR to stimulate SERT activity. In the CNS, adenosine is produced from at least three different sources. The most important source is believed to be the hydrolysis of adenosine 5′-monophosphate (5′-AMP) by 5′-nucleotidase. Extracellular 5′-AMP is derived partly from ATP, which is co-localized and co-released with other neurotransmitters including ACh, NE, and DA (Burnstock, 1990; Salter et al., 1993). An additional source of adenosine arises as a by-product of transmethylation reactions that are important for catabolism of catecholamines and histamine (via catecholamine O-methyltransferase and histamine N-methyltransferase). A third source involves the release of adenosine from neuronal and glial cells (Meghji and Newby, 1990; Nagy and Staines, 1990), possibly by reversal of the transporters normally responsible for adenosine uptake. Adenosine in the CNS generally exerts a tonic inhibitory effect on neural excitability (Prince and Stevens, 1992) primarily via A₁ and A_{2A}ARs. The concentration of adenosine and location of production are likely to be critical determining factors in the activation of AR subtypes. At low concentrations (nM range), adenosine activates A₁AR and A₂AAR; at higher concentrations (1M range), adenosine activates A_{2B} and A₃AR (Daly and Padgett, 1992; Zhou et al., 1992; Peakman and Hill, 1994). Therefore, conditions that cause pronounced release of ATP/adenosine may result in stimulation of SERT activity via A₃ARs.

In summary, this study provides the first evidence that A₃AR activation stimulates SERT function in the brain. Moreover, establish for the first time a conservation of SERT regulatory pathways from mast cells to brain 5-HT neurons involving A₃ARs, with important implications for the further mechanistic dissection of these processes. As a modulator of SERT function, inspection of the A₃AR gene for polymorphic variants may reveal genetic contributions to risk for mood disorders and/or antidepressant response. Additionally, the results suggest that pharmacological targeting of A₃ARs may represent a viable route for development of novel 5-HT modulatory therapeutics. Specifically, agents that block A₃ARs may be able to selectively diminish elevations in limbic SERT activity without impacting basal 5-HT clearance and thereby limit 5-HT clearance in a context-dependent manner that could be useful for the treatment of anxiety and depression.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. References

The following references, to the extent that they provide exemplary procedural or other details supplementary to those set forth herein, are specifically incorporated herein by reference:
U.S. Pat. No. 6,358,964
U.S. Pat. No. 6,407,236
U.S. Pat. No. 6,448,253
U.S. Pat. No. 6,673,802
U.S. Pat. No. 6,914,053
U.S. Pat. No. 6,921,825
U.S. Publn. 2003/0073708
U.S. Publn. 2003/0078232
U.S. Publn. 2003/0143282
U.S. Publn. 2003/0144266
U.S. Publn. 2004/0067932
U.S. Publn. 2004/0106572
U.S. Publn. 2004/0121978
U.S. Publn. 2004/0204481
U.S. Publn. 2005/0119289
U.S. Publn. 2005/0234056
U.S. Publn. 2005/0250729
U.S. Publn. 2006/0040959
U.S. Publn. 2006/0052331
U.S. Publn. 2006/0178385
Albasanz et al., Biochim. Biophys. Acta, 1593:69-75, 2002.
Ansah et al., J. Pharmacol. Exp. Ther., 305:956-965, 2003.
Ashton et al., Circulation, 76(4):952-959, 1987.
Barker and Blakely, In: Norepinephrine and serotonin transporters: molecular targets of antidepressant drugs, Raven Press, Ltd., NY, 1995.
Barnes, Science, 239(4842):864-866, 1988.
Bauman et al., J. Neurosci., 20:7571-7578, 2000.
Bengel et al., Mol. Pharmacol., 53:649-655, 1998.
Blackman et al., Biophys. J., 71(1):194-208, 1996.
Blakely et al., Nature, 354:66-70, 1991.
Blakely et al., Physiology (Bethesda), 20:225-231, 2005.
Blundell, Appetite, 7(Suppl):39-56, 1986.
Briley et al., Trends Pharmacol. Sci., 14(11):396-397, 1993.
Burnstock, Neurochem. Int., 17:357-368, 1990.
Carneiro and Blakely, J. Biol. Chem., 281:24769-24780, 2006.
Carrasco and Sandner, Int. J. Clin. Pract., 59:1428-1434, 2005.
Caspi et al., Science, 18:301(5631):291-293, 2005.
Caspi et al., Science, 301:386-389, 2003.
Daly and Padgett, Biochem. Pharmacol., 43:1089-1093, 1992.
Daws and Toney, In: High-speed chronoamperometry to study kinetics and mechanisms for serotonin clearance in vivo, FL, CRC Press, Taylor & Francis Group, 2006.
Daws et al., J. Neurochem., 75:2113-2122, 2000.
Dixon et al., Br. J. Pharmacol., 118:1461-1468, 1996.
Dostmann et al., Proc. Natl. Acad. Sci. USA, 97:14772-14777, 2000.
Dunwiddie et al., J. Neurosci., 17:607-614, 1997.
Fedorova et al., Cell Mol. Neurobiol., 23:431-447, 2003.
Feoktistov and Biaggioni, Pharmacol. Rev., 49:381-402, 1997.
Fredholm et al., Annu. Rev. Pharmacol. Toxicol., 45:385-412, 2005.
Fredholm et al., Pharmacol. Rev., 53:527-552, 2001.
Fredholm, Drug News Perspect., 16:283-289, 2003.
Gallo-Rodriguez et al., J. Med. Chem., 37:636-646, 1994.
Gu et al., J. Biol. Chem., 269:7124-7130, 1994.
Halldner et al., Neuropharmacology, 46:1008-1017, 2004.
Hoffman et al., Science, 254:579-580, 1991.
Holmes et al., Biol. Psychiatry, 54:953-959, 2003.
Huang and Pickel, J. Neurocytol., 31:667-679, 2002.
Jacobson et al., FEBS Lett., 336:57-60, 1993.
Jayanthi and Ramamoorthy, Aaps J, 7:E728-738, 2005.
Jayanthi and Ramamoorthy, J. Biol. Chem., 279:19315-19326, 2004.
Jayanthi et al., Mol. Pharmacol., 67:2077-2087, 2005.
Jennings et al., J. Neurosci., 26:8955-8964, 2006.
Koella, In: Sleep Disorders: Diagnosis and Treatment, 2^{nd} Ed., Wiley & Sons, NY, 1988.
Launay et al., Am. J. Physiol., 266:R526-536, 1994.
Le Bars, Pain, 32(2):259-261, 1988.
Lesch et al., Science, 274(5292):1527-1531 1996.
Loder and Melikian, J. Biol. Chem., 278:22168-22174, 2003.
Maemoto et al., Br. J. Pharmacol., 122:1202-1208, 1997.
Meghji and Newby, Neurochem. Int., 16:227-232, 1990.
Meltzer et al., Arch. Gen. Psychiatry, 38(12):1322-1326, 1981.
Miller and Hoffman, J. Biol. Chem., 269:27351-27356, 1994.
Miner et al., J. Comp. Neurol., 427:220-234, 2000.
Murphy et al., Genes Brain Behav., 2:350-364, 2003.
Murphy et al., Mol. Interv., 4:109-123, 2004.
Murrison et al., FEBS Lett., 384:243-246, 1996.
Nagy and Staines, Neurochem. Int., 16:211-221, 1990.
Naranjo et al., Clin. Pharmacol. Ther., 41(3):266-274, 1987.
Okada et al., Eur. J. Neurosci., 11: 1-9, 1999.
Owens and Nemeroff, Depress Anxiety, 8(1):5-12, 1998.
Ozaki et al., Mol. Psychiatry, 8:933-936, 2003.
Parsons et al., Faseb J, 14:1423-1431, 2000.
Paul et al., Arch. Gen. Psychiatry, 38(12):1315-13157, 1981.
Paxinos and Watson, In: The Rat Brain in Stereotaxic Coordinates, Academic Press, NY, 1986.
Peakman and Hill, Br. J. Pharmacol., 111:191-198, 1994.
Perry et al., Br. J. Psychiatry, 142:188-192, 1983.
Pickel and Chan, J. Neurosci., 19:7356-7366, 1999.
Prince and Stevens, Proc. Natl. Acad. Sci. USA, 89:8586-8590, 1992.
Qian et al., J. Neurosci., 17:45-57, 1997.
Quick, Int. J. Dev. Neurosci., 20:219-224, 2002.
Ramamoorthy and Blakely, Science, 285:763-766, 1999.
Ramamoorthy et al., J. Biol. Chem., 268(29):21626-21631, 1993.
Ramamoorthy et al., Proc. Natl. Acad. Sci. USA, 90:2542-2546, 1993.
Ramkumar et al., J. Biol. Chem., 268:16887-16890, 1993.
Sajjadi and Firestein, Biochim. Biophys. Acta, 1179:105-107, 1993.
Salter et al., Prog. Neurobiol., 41:125-156, 1993.
Salvatore et al., J. Biol. Chem., 275:4429-4434, 2000.
Salvatore et al., Proc. Nat. Acad. Sci. USA, 90:10365-10369, 1993.
Samuvel et al., J. Neurosci., 25:29-41, 2005.
Schloss, J. Psychopharmacol., 12(2):115-121, 1998.
Scott et al., Proc. Natl. Acad. Sci. USA, 102:16472-16477, 2005.
Serebruany, Am. J. Med., 119:113-116, 2006.
Silverstone et al., Appetite, 7(Suppl):85-97, 1986.
Suranyi-Cadotte et al., Life Sci., 37(24):2305-2311, 1985.
Sutcliffe et al., Am. J. Hum. Genet., 77:265-279, 2005.
Talukder et al., Am. J. Physiol. Heart Circ. Physiol., 282:H2183-2189, 2002.
Taylor et al., Mol. Pharmacol., 65:1111-1119, 2004.
Van Woert et al., Monogr. Neural. Sci., 3:71-80, 1976.
Yaar et al., J. Cell Physiol., 202:9-20, 2005.
Zhou et al., Proc. Nat. Acad. Sci. USA, 89:7432-7436, 1992.
Zhou et al., Proc. Natl. Acad. Sci. USA, 89:7432-7436, 1992.
Zhu et al., Eur. J. Pharmacol., 504:1-6 2004b.
Zhu et al., J. Biol. Chem., 280:15649-15658, 2005.
Zhu et al., Mol. Pharmacol., 65:1462-1474, 2004a.

Zhu et al., Mol. Pharmacol., 65:1462-1474, 2004a.
Zhu et al., Neuropsychopharmacology, 31:2121-2131, 2006.

What is claimed is:

1. A method of modulating serotonin transporter (SERT) function in a native brain preparation or tissue comprising introducing into said preparation or tissue an antagonist of adenosine type 3 receptor (A3R) function.

2. The method of claim 1, wherein said antagonist is an A3R antagonist that reduces A3R-dependent increases in SERT function.

3. The method of claim 2, wherein said antagonist is MRS-1191, MRS-1523 or VUF5574.

4. The method of claim 2, further comprising titrating said antagonist to optimize inhibition.

5. The method of claim 4, wherein optimizing inhibition comprises preventing upregulation of SERT function without blocking basal SERT function.

6. The method of claim 1, further comprising measuring 5-HT uptake in said preparation or tissue prior to introducing said A3R antagonist.

7. The method of claim 1, further comprising measuring 5-HT uptake in said preparation or tissue at the time of and/or subsequent to introducing said A3R antagonist.

8. The method of claim 1, wherein said antagonist is contacted with said preparation or tissue more than once.

9. The method of claim 1, further comprising introducing into said preparation or tissue a second SERT antagonist.

10. The method of claim 1, wherein said preparation or tissue is in a subject.

11. The method of claim 10, wherein said subject is a human subject.

12. The method of claim 10, wherein said antagonist is delivered orally.

13. The method of claim 10, wherein said antagonist is administered via subcutaneous, intraspinal or intravenous injection.

14. The method of claim 1, wherein said antagonist is administered on a chronic basis.

15. The method of claim 10, wherein subject suffers from depression, an anxiety disorder, obsessive compulsive disorder or autism.

16. A method of desensitizing serotonin transporter (SERT) function in a native brain preparation or tissue comprising chronic treatment of said preparation or tissue with an A3 agonist to enhance SERT activity.

17. The method of claim 16, wherein said A3 agonist is IB-MECA, CF101, AB-MECA or N6-2-(4-Aminophenyl)ethyladenosine.

18. The method of claim 16, further comprising measuring 5-HT uptake in said preparation or tissue prior to introducing said A3 agonist.

19. The method of claim 16, further comprising measuring 5-HT uptake in said preparation or tissue at the time of and/or subsequent to introducing said A3 agonist.

20. The method of claim 16, further comprising introducing into said preparation or tissue a second A3 agonist.

21. The method of claim 16, wherein said preparation or tissue is in a subject.

22. The method of claim 21, wherein said subject is a human subject.

23. The method of claim 21, wherein said agonist is delivered orally.

24. The method of claim 21, wherein said agonist is administered via subcutaneous, intraspinal or intravenous injection.

25. The method of claim 21, wherein subject suffers from depression, an anxiety disorder, obsessive compulsive disorder or autism.


SEQUENCE LISTING (2 sequences)

SEQ ID NO: 1 - DNA, 2756 bp, Homo sapiens
ccgccgggtgcctcgagggcgcgaggccagcccgcctgcccagcccgggaccagcctccccgcgcagcctggcaggtctcctggaggcaaggcgaccttgcttgccctctattgcagaataacaaggggcttagccacaggagttgctggcaagtggaaagaagaacaaatgagtcaatcccgacgtgtcaatcccgacgatagagagctcggaggtgatccacaaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggggcattcctcctcccctacaccatcatggccatttttgggggaatcccgctcttttacatggagctcgcactgggacagtaccaccgaaatggatgcatttcaatatggaggaaaatctgcccgattttcaaagggattggttatgccatctgcatcattgccttttacattgcttcctactacaacaccatcatggcctgggcgctatactacctcatctcctccttcacggaccagctgccctggaccagctgcaagaactcctggaacactggcaactgcaccaattacttctccgaggacaacatcacctggaccctccattccacgtcccctgctgaagaattttacacgcgccacgtcctgcagatccaccggtctaaggggctccaggacctggggggcatcagctggcagctggccctctgcatcatgctgatcttcactgttatctacttcagcatctggaaaggcgtcaagacctctggcaaggtggtgtgggtgacagccaccttcccttatatcatcctttctgtcctgctggtgaggggtgccaccctccctggagcctggaggggtgttctcttctacttgaaacccaattggcagaaactcctggagacaggggtgtggatagatgcagccgctcagatcttcttctctcttggtccgggctttggggtcctgctggcttttgctagctacaacaagttcaacaacaactgctaccaagatgccctggtgaccagcgtggtgaactgcatgacgagcttcgtttcgggatttgtcatcttcacagtgctcggttacatggctgagatgaggaatgaagatgtgtctgaggtggccaaagacgcaggtcccagcctcctcttcatcacgtatgcagaagcgatagccaacatgccagcgtccactttctttgccatcatcttctttctgatgttaatcacgctgggcttggacagcacgtttgcaggcttggagggggtgatcacggctgtgctggatgagttcccacacgtctgggccaagcgccgggagcggttcgtgctcgccgtggtcatcacctgcttctttggatccctggtcaccctgacttttggaggggcctacgtggtgaagctgctggaggagtatgccacggggcccgcagtgctcactgtcgcgctgatcgaagcagtcgctgtgtcttggttctatggcatcactcagttctgcagggacgtgaaggaaatgctcggcttcagcccggggtggttctggaggatctgctgggtggccatcagccctctgtttctcctgttcatcatttgcagttttctgatgagcccgccacaactacgacttttccaatataattatccttactggagtatcatcttgggttactgcataggaacctcatctttcatttgcatccccacatatatagcttatcggttgatcatcactccagggacatttaaagagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatataattttattgctggaatctactagaaccttctaatccatgtgctgctgtggcatcaggaaaggaagatgtaagaagctaaaatgaaaaatagtgtgtccatgcaa

SEQ ID NO: 2 - protein, 630 aa, Homo sapiens
METTPLNSQKQLSACEDGEDCQENGVLQKVVPTPGDKVESGQISNGYSAVPSPGAGDDTRHSIPATTTTLVAELHQGERETWGKKVDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSEDNITWTLHSTSPAEEFYTRHVLQIHRSKGLQDLGGISWQLALCIMLIFTVIYFSIWKGVKTSGKVVWVTATFPYIILSVLLVRGATLPGAWRGVLFYLKPNWQKLLETGVWIDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHVWAKRRERFVLAVVITCFFGSLVTLTFGGAYVVKLLEEYATGPAVLTVALIEAVAVSWFYGITQFCRDVKEMLGFSPGWFWRICWVAISPLFLLFIICSFLMSPPQLRLFQYNYPYWSIILGYCIGTSSFICIPTYIAYRLIITPGTFKERIIKSITPETPTEIPCGDIRLNAV